(19) United States
(12) Patent Application Publication
(10) Pub. No.: US 2011/0131171 A1
(43) Pub. Date: Jun. 2, 2011

(54) RISK STRATIFICATION OF GENETIC DISEASE USING SCORING OF AMINO ACID RESIDUE CONSERVATION IN PROTEIN FAMILIES

(75) Inventors: Christian Jons, Rochester, NY (US); Arthur Moss, Rochester, NY (US); Coeli Lopes, Rochester, NY (US); Scott McNitt, Rochester, NY (US)

(73) Assignee: University Of Rochester, Rochester, NY (US)

(21) Appl. No.: 12/989,090

(22) PCT Filed: Apr. 24, 2009
(86) PCT No.: PCT/US09/41663

§ 371 Date: Jan. 26, 2011

Related U.S. Application Data
(60) Provisional application No. 61/047,593, filed on Apr. 24, 2008.

(51) Int. Cl. C12Q 1/68; G01N 33/50; G06N 5/02; G06F 17/30

(52) U.S. Cl. 706/54; 435/6.1; 436/94; 707/748

(57) ABSTRACT

Methods, databases and software for determining the risk of an adverse health event for patient by analysis of the protein sequence of the patient are described. The methods involve obtaining a protein sequence that is associated with a specific disorder from the patient. The protein sequence from the patient is compared to a database of sequences of the same protein and is analyzed to determine the conservation score of the amino acid residues in the protein. Those amino acid residues having high conservation scores will be further analyzed to determine if there are mutations present at those highly conserved positions. Patients having proteins with mutations in highly conserved positions are determined to have a higher risk of an adverse event due to the disorder.

### FIELD OF THE INVENTION

The present invention relates to the risk stratification of patients having disorders with an underlying genetic basis. More specifically, the present invention relates to the analysis of the genetic sequences of patients for the purposes of determining the patients' risk of adverse events from a specific disorder.

### BACKGROUND OF THE INVENTION

Risk stratification is frequently used by doctors to analyze patient risks of adverse events in order to better determine treatment options. Various factors associated with a specific disorder can be analyzed and used to determine a patient's risk. After the risk is determined, the patient can be presented with various prevention and treatment options suitable for someone with their level of risk of an adverse event due to a disorder.

Most factors used by doctors in performing a risk stratification analysis are either general, including age, gender, and family history, or clinical signs that are typically associated with a specific disorder. Specific clinical signs vary widely depending on the analysis, but may include unusual cardiograms for patients at risk of cardiovascular events or signs of unusual blood chemistry or the presence of biomarkers in the fluids of patients at risk of developing a specific type of cancer.

Risk stratification using general or clinical factors, while helpful, only provides a modest estimate of the real risk to the patient of a life-threatening adverse event. General factors such as age and gender are, of course, not specific to the specific disorder being investigated and, as such, only provide general guidance. Clinical factors may in some cases be more specific, but may also be indicators of other problems. Also, by the time some clinical factors are detectable, the patient may already have begun developing the life-threatening effects about which the doctor is concerned.

Continuing advances in genome sequencing and analysis have allowed for the rapid determination and comparison of genomic information of many organisms, especially humans. Analysis and comparison of organisms on the genomic level has allowed scientists to pinpoint specific regions of proteins that are related to the pathogenesis of genetic diseases and disorders. Further, the ability to rapidly obtain and compare genetic information from different subjects has allowed for the determination of conserved regions of amino acids, regions that are almost always important for the function of the protein. Comparison of genomic sequences through alignment and comparison algorithms allows for the determination of important amino acid residues without the need for in-depth biochemical study.

Although genomic analysis has become abundant, facile and efficient methods for correlating genomic analysis with clinical risk determination have not been developed. There remains a need in the art for methods that allow for the improvement of clinical risk stratification through the use of genomic analysis.

### SUMMARY OF THE INVENTION

It is an object of the present invention to provide a method for correlating genetic sequence information with the risks of a patient having an adverse health event. The present invention provides a method for comparing genomic sequences from different sources and determining which areas of the genomic sequence are conserved between the compared sequences. Genomic sequences having mutations in highly conserved regions will correlate with favorable and unfavorable outcomes of the patient having that sequence. As multiple sequences are analyzed, the correlation of mutations in conserved regions of sequences and clinical outcomes will allow for the accurate prediction of risks of adverse events.

It is a further object of the present invention to provide a method for determining the risk of an adverse event for a patient through the analysis of the patient's genomic information. The method involves obtaining a genomic sample from the patient and determining its sequence. The sequence of a specific genetic region of the patient associated with the adverse event is analyzed for mutations in conserved regions. The presence of mutations in conserved regions of the genomic sequence of the patient can then be used to determine the patient's risk of an adverse health event.

It is a still further object of the present invention to provide a database that can be used in determining the risk of an adverse event for a patient by analysis of mutations in conserved regions of a genomic sequence of the patient. The database will be able to be expanded continually with new genomic information and risk data, allowing for increased accuracy of risk determination as the database expands.

It is a still further object of the present invention to provide a method for creating databases that can be used in determining the risk of an adverse event for a patient by analysis of a genomic sequence of the patient. The databases of the present invention are assembled through the comparison, alignment and analysis of multiple genomic sequences. The presence or absence of mutations in conserved regions of the genomic sequences are associated with the clinical outcome of the patients having those differences, which allows for the correlation of genomic data and the risk of adverse events.

It is yet a further object of the present invention to provide software for the analysis of a patient's risk of an adverse event based on the presence of mutations within a genomic sequence of the patient. The software allows for the input of the genomic sequence to be analyzed. The software then compares the inputted genomic sequence with those in a database of sequences, and provides the patient's risk of an adverse event.

### BRIEF DESCRIPTION OF THE DRAWINGS

FIG. 1 shows a sample of the multiple sequence alignment from the pore region of the KCNQ1 channel (SEQ ID NO: 38), amino acid residue number 300 through 324, a region where the degree of conservation of the amino acid residues is generally high.

FIG. 2 shows the location of mutations in a schematic diagram of the KCNQ1 channel from amino acid residue 117 through 374 by tertile of the adjusted Shannon entropy score.

FIG. 3 shows a plot of Kaplan-Meier estimates of the cumulative probability of first cardiac event (A) and first aborted cardiac arrest or sudden cardiac death (B) from birth to age 41 years by tertiles of the adjusted Shannon entropy score. Both models are adjusted for patients who died before having an ECG recorded (QTc missing), but this parameter is not shown in the tables (see text).

### DETAILED DESCRIPTION OF THE INVENTION

The present invention provides methods, databases and software for the determination of a patient's risk of a favorable and adverse health event through the analysis of a genomic sequence of the patient. In determining the patient's risk, other risk stratification factors, such as age, gender, family history, and clinical symptoms, may be used as part of the analysis. The methods of the present invention may also be used on their own to analyze patient risk. The present invention provides the tools for more accurate risk determination by analyzing factors (e.g. genomic sequences) that are closely related to the risk being determined.

The methods, databases, and software can be used for associating the risk of adverse health events for various disorders, diseases, syndromes and the like. Throughout the specification, these terms will be used interchangeably, and it should be apparent to one of skill in the art that any embodiment of the present invention which is applicable to use for risk determination for a disorder can also be used for risk determination for a disease, syndrome or other ailment.

In one embodiment of the present invention, methods are provided for associating the degree of conservation of a mutation in a genomic sequence with the risk of an adverse health event. The genomic sequences to be compared are determined by the disorder for which an adverse event is being determined. In many cases, a specific protein, gene or locus is associated with the disorder at issue. The part of the patient's genome that corresponds to this protein, gene or locus can then be used as the genomic sequence of interest. Typically, the genomic sequence being analyzed will be a protein sequence, however it is contemplated that the genomic sequence could be a nucleic acid sequence. For the purposes of this description, comparison of protein sequences will be used as the primary example.

Geneotypes for the mutations characterized may be identified using standard genetic tests as are well known in the art. Mutations for analysis may be determined from previous studies or may be genotyped using well known methods. For examples, genotypes in a protein known to be associated with a disorder may be used to determine specific mutations which will be analyzed to determine a conservation score.

Any reliable phenotype associated with a specific disorder may be used for correlation with a conservation score as determined by the methods of the present invention. Phenotypes such as clinical manifestations of a disorder or diagnostic indicators such as biomarkers may be determined using methods well known in the art. For example, clinical manifestations such as the presence of a malignancy may be used. Any type of biomarker may be used, such as the determination of increased levels of an antigen associated with a disorder.

Once a protein sequence of interest is identified, regions of conservation within the protein sequence are then identified. A number of representatives of the same protein sequence are collected from various sources. In certain embodiments of the present invention, it is preferred to have between 12-15 or more sequences for performing the alignment. However, it is also contemplated that fewer sequences can be used. Typically, as more sequences are added to the alignment, the degree of confidence in the risk stratification will increase. The sequences may be all from the same organism or may be from different organisms that have the proteins in the same family. Either type of sequence is considered to be a related protein for purposes of the present invention.

Protein sequences appropriate for alignment may be drawn from a number of databases well known in the art. In certain embodiments, sequences may be drawn from the Uniprot/Swissprot-database and aligned using the public sequence aligner CLUSTALW2.¹⁷ However, it is also contemplated that other databases and alignment tools, which are well known in the art, can be used in performing the present invention.

After a sufficient number of sequences are obtained, the degree of conservation for regions in individual sequences can be calculated. Typically, the degree of conservation is calculated for a specific amino acid residue, although it is also contemplated that the degree of conservation from a specific region up to the entire length of the protein could be used.

Determination of the degree of confirmation is often done through entropy calculations. In certain embodiments of the invention, the Shannon Entropy is used for entropy calculations, as originally described by Shannon¹⁶, the disclosure of which is hereby incorporated by reference herein. It is also contemplated that, in other embodiments of the present invention, the necessary characterization of the degree of conservation of a specific region can be calculated using other methods, such as the Von Neumann Entropy, the Property Entropy and the Jensen-Shannon diversity.¹⁷ A further description of methods for characterizing the conservation of sequences can be found in Valdar¹⁵, the disclosure of which is hereby incorporated by reference herein. It should be apparent to one of skill in the art that several methods allow for the determination of the conservation of a region of the protein sequence.

The Shannon entropy is defined as the following:

$$Shannon Entropy ( W ) = K \times {\sum }_{i = 0}^{20} ( p ( {x}_{i} ) \times ln p ( {x}_{i} ) )$$

Where W is a column in the multiple protein sequence alignment, x is an amino acid in the multiple protein sequence alignment, and i is a number between 0 and 20 corresponding to one of the 20 amino acid residues used in human proteins or an empty space. The probability of xᵢ is estimated from the frequency of the individual amino acid residue within the alignment column:

$$p ( {x}_{i} ) \approx f ( {x}_{i} ) = \frac{N ( {x}_{i} )}{L}$$

where N is the number of appearances of specific amino acid residue and L is the total number of amino acid residues in the current column of the alignment. K is a positive constant resealing the entropy to a number between 0 and 1, in this case defined as:

$$K = \frac{1}{ln ( i )}$$

A full conservation of one amino acid residue within the column will give a score of 0, whereas an alignment with no conservation will result in a score of 1. An online tool is available for calculation of the Shannon entropy,¹⁶ which has been used to score the sequences used in this study. In order to make scores comparable with other reported conservation scores, an adjusted Shannon entropy score may be used, i.e. 1-Shannon entropy, with 0 corresponding to no conservation and 1 to maximal conservation.

Once the conservation scores for each residue in the protein sequence are calculated, the protein sequences are correlated with the occurrence of phenotypes associate with the disorder. For instance, for protein sequences related to cardiac disorders, altered sequences can be correlated with adverse cardiac events such as syncope, cardiac arrest and cardiac death. The correlation between mutations in specific conserved positions and adverse events can then be used to determine risk to the patient of an adverse event.

Patients may be risk stratified according to the positions where they possess mutations in their protein sequences. For instance, if a patient only has a mutation at amino acid positions that have a conservation score indicating low conservation, the patient will be placed into a low risk group. By contrast, if the patient has mutations at positions that have conservation scores indicating high conservation, the patient will be placed in a high-risk group. The number of groups may vary and a number of different distinctions may be drawn. Although the value of a high conservation score will vary depending on the genetic factor analyzed, in some embodiments, high conservation scores may be considered to be scores having an adjusted Shannon entropy score of greater than about 0.50. In other embodiments, high conservation scores may be considered to be scores of greater than about 0.50 to greater than about 0.95, or any value inbetween.

Risk stratifications are formed by determining entropy score cutoff values. The cutoff values define the boundaries of the risk groups. The total number of risk groups may be varied as is necessary to give a stratification that allows for useful prediction. For example, the number of risk groups may be as low as 2 or as high as 12 or more. The cutoff values are typically defined so that the highest risk group contains members with a significant risk of developing the disorder. However, it is also possible that the cutoff values and number of groups may be defined so that there are two, three or even more groups that have a significant risk of developing the disorder. Groups may also be defined to determine those with different grades of moderate and low risk as is deemed necessary.

It is contemplated that patients may be risk stratified only on the basis of the presence of mutations at conserved amino acid residues. However, it is also contemplated that other risk stratification factors may also be used along with the conservation analysis, such as general or clinical factors.

After patients are stratified into groups, other indicators may be assigned to the group in order to help health care professionals communicate the patient's risk to them. Such mathematical approaches are well known in the art, and include biostatistical methods such as the hazard ratio. A demonstration of the use of hazard ratios in clinical trials is shown in Spruance et al.⁴⁰, the disclosure of which is hereby incorporated by reference herein.

It is further contemplated that the database of the present invention may be assembled without correlating the conservation scores to phenotypes such as clinical indications or biomarkers. In these embodiments of the present invention, the patients' risk may be assessed solely based on the presence of mutations at highly conserved amino acids.

All of the embodiments of the present invention are suitable for determining the risk of any disorder associated with a genetic factor. The genetic factors of the present invention are typically genes encoding functional proteins, including, but not limited to, channels, enzymes, transcription factors and regulatory. Any disorder associated with a gene encoding a protein for which conserved residues can be determined could be analyzed using the present invention.

The risk diagnosis of the present invention includes all classes of diseases, disorders, syndromes and other ailments which are associated with one or more mutations. The present invention is amenable to diagnosis of cardiac, neurological, respiratory, muscle, gastrointestinal and ocular syndromes and diseases, as well as disorders of other systems and organs of the body. The present invention is further amenable to diagnosis of genetically associated cancers and other malignancies.

Some non-limiting examples of disorders and certain genes that may be used for determining risk of developing a disorder are listed below. This brief list is not meant to be exhaustive, nor is it meant to include every single genetic factor that may be associated with a disorder. One of skill in the art will be able to apply the present invention to any disorder associated with a genetic factor for which one or more conserved residues may be determined.

Long QT-Syndrome—KCNQ1, KCNH2, SCN5A, ANK2, KCNE1, KCNE2, KCNJ2, CACNA1C, CAV3, SCN4B, and AKAP9.

Cystic fibrosis—CFTR.

Breast cancer—BRCA1, BRCA2, BRCATA, BRCA3, BWSCR1A, TP53 BRIP1, RB1CC1, RAD51, CHEK2, BARD1, PIK3CA, AKT1, PALB2, CASP8, TGFB1, NQO1, and HMMR.

Colon cancer—APC, MSH2, MLH1, PMS1, PMS2, MSH6, TFGBR2, MLH3, MUTYH, AXIN2, KRAS, PIK3CA, BRAF, CTNNB1, AXIN2, AKT1, MCC, MYH11 and SMAD7.

Lung cancer—EGFR, p53, KRAS, BRAF, ERBB2, MET, STK11, PIK3CA, EGFR, ERBB2, MET, P1K3CA, NKX2-1, ERCC6, CYP2A6, CASP8 and MPO.

Alzheimer disease—APP, APOE*4, PSEN1, PSEN2, A2M, LRP1, TF, HFE, NOS3, VEGF, ABCA2, and TNF.

Parkinson's disease—SNCA, UCHL1, LRRK2, HTRA2, SNCAIP, parkin, DJ1, HTRA2, LRRK2, NR4A2, NDUFV2, ADH3, FGF20, GBA, and MAPT.

Autism—NLGN3, NLGN4, MECP2, and GLO1.

Further examples of genetic factors associated with disorders for which risk can be determine using the present invention can be found in the Online Inheritance in Man (OMIM) database, which is available at http://www.ncbi.nlm.nih.gov/omim.

The databases of the present invention are typically embodied on a computer readable medium, and may be stored locally or on a server. The databases may be internet accessible or accessible through local networks.

In another embodiment of the present invention, methods are provided for determining the risk of an adverse event for a patient by analysis of a genomic sequence of a patient. The methods of the present invention involve obtaining genomic information for a patient, analyzing the genomic sample, and using the results of the analysis to determine the risk to the patient of developing a disorder.

In a first step of the methods of the present invention, a genomic sequence is obtained from the patient. In many cases, there may be more than one applicable genomic sequence for a specific disorder, and some or all of these sequences may be used in the risk analysis.

Typically, a genomic sequence is obtained by collecting a body fluid or tissue sample from the patient, isolating the nucleic acid, and obtaining the nucleic sequence from the patient, as is well known in the art. Examples of body fluid or tissue samples include blood, saliva, cells, semen, cerebro-spinal fluid, aqueous humor, mucus, sweat, pus, sebum, tissue section, biopsy samples and the like. It should be apparent to one of skill in the art that a patient sample may be obtained by a person or entity that did not collect the sample. For example, if a testing laboratory receives a patient sample for nucleic acid isolation, the testing laboratory has obtained the sample within the meaning of the present disclosure.

It is also possible that a complete or partial genome sequence is already known for the patient. In these cases, the genomic sequence of interest can be obtained from the information already available, without the need for taking a patient fluid or tissue sample.

Once the nucleic acid sequence is obtained, it is typically converted into a protein sequence made up of amino acids. The protein sequence sample is then compared to previously known sequences of the same type per the analysis described above. The patient may then be associated with a specific risk group according to the presence or absence of mutations in conserved amino acid residues of the protein sequence being analyzed.

In other embodiments of the present invention, databases and methods for making them are provided. A database will typically be associated with a specific genome sequence for analysis, such as a protein. The database will contain all of the amino acid sample sequences for that protein, will identify conserved amino acid residues and will correlate those conserved residues with adverse events as described above. The databases of the present invention are meant to be expandable. Typically, when a new sequence is analyzed, this sequence will be added to the database as a reference sequence. The database may also allow for the updating of information about adverse events or other risk factors, allowing new information to be associated with sequences already in the database. By continuing to add more sequence and risk information to the database, the accuracy of the risk analysis will continue to improve.

In another embodiment of the present invention, software is provided that performs the risk analysis. When a patient's genomic sequence is obtained, as described above, it may be entered into the software. The software is designed to access a database as described and perform the risk analysis of the present invention, outputting the risk so that the doctor or other medical professional may inform the patient. It is contemplated that the software of the present invention may be software stored on a local computer, or may alternatively be server or web-based, allowing for its access from remote computers.

It is contemplated that all or some of the steps of the methods of the present invention may be performed by specialized laboratories. These laboratories may receive patient samples, isolate and analyze sequence information and return the risk analysis results to the medical professional. In this scenario, the specialized laboratory may be capable of developing large databases for a number of disorders, and will allow medical professionals to obtain this type of risk analysis without the need to perform the methods of the invention themselves.

Specific examples of risk analysis are given in the examples below. Although the examples focus on specific disorders, it should be apparent to one of skill in the art that the present invention is readily adaptable to almost any disorder, disease or syndrome in which a genetic factor is known.

### EXAMPLES

### Example 1

### Risk Stratification in Long-QT Syndrome

### Introduction:

Type-1 long-QT syndrome (LQT1) is caused by loss-of-function mutations in the KCNQ1-gene encoding the KCNQ1 channel alpha subunit.¹ The channel is responsible for the slowly activating late repolarizing potassium current in the human heart. The gene encoding the KCNQ1 subunit was cloned for the first time in 1996,² and today more than three hundred different LQT1-related mutations have been identified in this gene.³ The KCNQ1 channel is a member of the voltage-gated potassium channel (K_{V}) family. In this family, four KCNQ1 subunits oligomerize with beta-subunits to form the channel. The KCNQ1 subunit structure includes an N-terminus, six membrane-spanning domains (S1 through S6) and a C-terminus. The 3-dimensional structure of a related potassium channel has been reported,⁴ and recently a suggested model structure of the KCNQ1 channel has been published.⁵ The six membrane spanning domains are thought to have distinct functions, with S1-S4 forming a voltage-gating domain, S5-S6 forming the ion conduction pathway and N- and C-terminal areas being important in intracellular signaling.⁶

Patients with LQT1 are at increased risk of recurrent syncope and cardiac death due to arrhythmias.⁷ However, the occurrence of syncope and cardiac arrest is quite variable within the clinical syndrome, and proper risk stratification is needed in order to optimize patient treatment.⁷⁻¹² The purpose of this study was to investigate whether missense mutations in highly conserved amino acids of the KCNQ1 channel are associated with a more virulent clinical course than mutations in other amino acids. It was hypothesized that bio-informational methods used to identify conserved amino acid residues in protein sequences by conservation analysis would identify mutations associated with an increased risk of cardiac events in patients with the LQT1 genotype.

### Methods

### Population

The study population (n=492) was drawn from the U.S. portion of the International LQTS Registry (n=361), the Netherlands' LQTS Registry (n=55), and the Japanese LQTS Registry (n=47) as previously reported,⁸ plus additional patients from the Danish Registry (n=29). Subjects were included if they had a genetically confirmed missense mutation located within the area of the KCNQ1 gene defined below or if they had died suddenly at a young age and were from a family with such a mutation. Patients were excluded from the study if they suffered from hearing loss indicative of the Jervell and Lange-Nielsen syndrome or had multiple mutations. Only patients with missense mutations were included in this study. All subjects or their guardians provided informed consent for the genetic and clinical studies.

The area of the KCNQ1 channel that shows a high homology and conservation within the human K_{V}-family was studied, and the domains within the KCNQ1 channel by homology with the Kv1.2 channel, for which the crystal structure have been published were defined.⁴ The conserved channel region included in this study comprised the 5 residues of the N-terminus closest to the S1 domain, the membrane-spanning domains S1-S6 including linkers, and the proximal 17 amino acid residues of the C-terminus. Patients with mutations within this region, in amino acid residues 117-374, were included in the study. Amino acid residues outside this region showed too low homology among the K_{V} channel family members to be aligned. A total of 722 patients with genotyped mutations were identified in the 4 registries. Patients with 2 or more mutations (n=28), 128 patients with non-missense mutations, 74 with missense mutations located outside the aligned region, and 6 patients with insufficient survival data were excluded. The study population Involved 492 patients with KCNQ1 missense mutations.

### Genotype Characterization

The KCNQ1 mutations were identified with the use of standard genetic tests performed in academic molecular-genetic laboratories including the Functional Genomics Center, University of Rochester Medical Center, Rochester, N.Y.; Baylor College of Medicine, Houston, Tex.; Mayo Clinic College of Medicine, Rochester, Minn.; Boston Children's Hospital, Boston, Mass.; Laboratory of Molecular Genetics, National Cardiovascular Center, Suita, Japan; Department of Clinical Genetics, Academic Medical Center, Amsterdam, Netherlands; and Statens Seruminstitut, Copenhagen, Denmark.

### Phenotype Characterization

The ECG parameters were obtained from the baseline ECG recorded at the time of patient enrollment in each of the registries. The QT and R-R intervals were measured in milliseconds, with QT corrected for heart rate by Bazett's formula (QTc). Follow-up was censored at age 41 years to avoid the influence of coronary and other late-onset diseases on cardiac events. In all 4 registries, clinical data were collected on prospectively designed forms for demographic characteristics, personal and family medical history, ECG findings, therapy, and end-points during long-term follow-up. Data common to all 4 LQTS registries involving genetically identified patients with LQT1 genotype were electronically merged into a common database for the present study.

### The Conservation Score

Characterization of a protein by determination of functional areas can be done by bio-informational analysis of multiple protein-sequence alignments.^{13, 14} The analysis most frequently employed categorizes the entropy of aligned amino acid residues utilizing a mathematical approach originally described by Shannon.¹⁵ The Shannon entropy is defined as the following:

$$Shannon Entropy ( W ) = K \times {\sum }_{i = 0}^{20} ( p ( {x}_{i} ) \times ln p ( {x}_{i} ) )$$

Where W is a column in the multiple protein sequence alignment, x is an amino acid in the multiple protein sequence alignment, and i is a number between 0 and 20 corresponding to one of the 20 amino acid residues used in human proteins or an empty space. The probability of xᵢ is estimated from the frequency of the individual amino acid residue within the alignment column:

$$p ( {x}_{i} ) \approx f ( {x}_{i} ) = \frac{N ( {x}_{i} )}{L}$$

where N is the number of appearances of specific the amino acid residue and L is the total number of amino acid residues in the current column of the alignment. K is a positive constant resealing the entropy to a number between 0 and 1, in this case defined as:

$$K = \frac{1}{ln ( i )}$$

A full conservation of one amino acid residue within the column will give a score of 0, whereas an alignment with no conservation will result in a score of 1. An online tool is available for calculation of the Shannon entropy,¹⁶ which has been used to score the sequences used in this study. In order to make scores comparable with other conservation scores, conservation is reported as an adjusted Shannon entropy score, i.e. 1-Shannon entropy, with 0 corresponding to no conservation and 1 to maximal conservation.

### Protein Selection and Alignment

Protein sequences appropriate for alignment were drawn from the Uniprot/Swissprot-database and aligned using the public sequence aligner CLUSTALW2.¹⁷ The multiple protein sequence alignment was made using sequences for all 38 known human channels belonging to the voltage-gated potassium channel family (K_{V}-family). Since sequences within this family show a low degree of similarity in certain areas of the gene, regions for subunits S1, S2, S3, 54, and S5-pore-S6 region where aligned individually and co-assembled afterwards into a continuous sequence relative to the KCNQ1 sequence.

### Endpoints

LQTS-related cardiac events included syncope, aborted cardiac arrest, and sudden cardiac death (unexpected sudden death without a known cause). Information on end-point events was determined from the clinical history ascertained by routine follow-up contact with the patient, family members, attending physician, or the medical records. Categorization of the end point was based on pre-specified criteria.⁸

### Statistics

Standard statistical tests were utilized in the univariate comparison analyses. The cumulative probability of a first cardiac event was assessed by the Kaplan-Meier method with significance testing by the log-rank statistic. The Cox proportional hazards survivorship model was used to evaluate the independent contribution of clinical and genetic factors to the first occurrence of time-dependent cardiac events from birth through age 40 years. The influence of gender as a covariate is not proportional as a function of age with crossover in risk at age 13 years on univariate Kaplan-Meier analysis. Gender was therefore modeled in an unstratified Cox model as a time-dependent covariate (via an interaction with time), allowing for different hazard ratios by gender before and after age 13 years. Categorization of data into tertiles was pre-specified, but since the distribution of the adjusted Shannon entropy score is affected by the differing number of family members, the distribution shows some non-linearity and tertiles are not of equal population size.

Since almost all the subjects were first- and second-degree relatives of probands, the robustness of the findings was tested using several methods. Because of a potential lack of independence between subjects, the Cox model was fit using the robust sandwich estimator for family membership.¹⁸ To explore the functional form of the relationship between the Shannon entropy score and the endpoint, quartiles, quintiles and octiles were also fit in the multivariate Cox models. The pattern of the relationship was consistent among these various analyses. Additionally, to assure that the reported results were not due to inequalities in family size, Cox models were fit incorporating weights equal to the inverse number of family members. The results from the weighted models were consistent with those from the unweighted models. Since the unweighted model results displayed robustness to family size and membership, they are presented here.

Patients who died suddenly at a young age from suspected LQTS and who did not have an ECG for QTc measurement were identified in the Cox models as “QTc missing”. Pre-specified covariate interactions were evaluated. The influence of time-dependent beta-blocker therapy (the age at which beta-blocker therapy was initiated) on outcome was included in the Cox model.

### Results

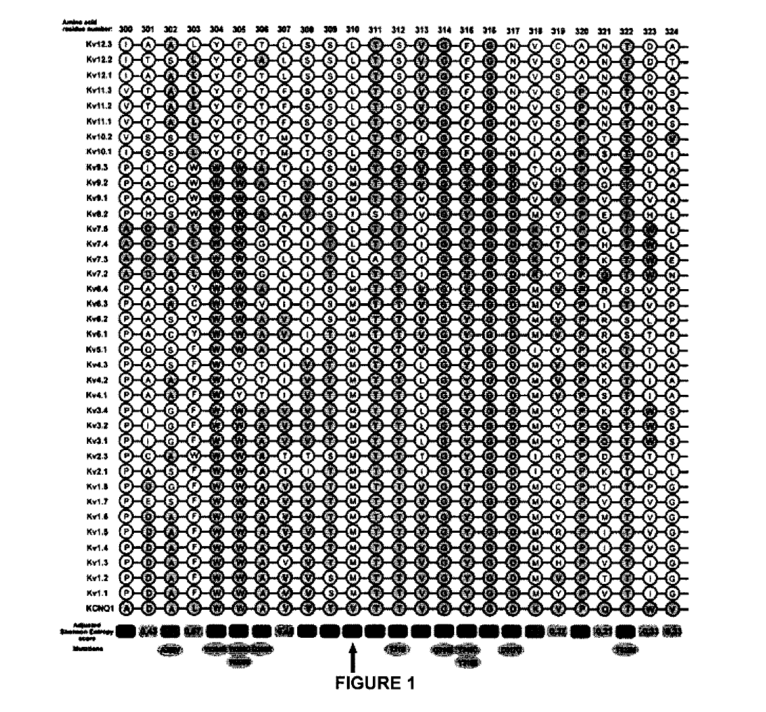

The study population involved 492 genotyped LQT1 patients with 54 different missense mutations. FIG. 1 shows a sample of the amino acid alignment with the adjusted Shannon entropy scores under each alignment column. The figure shows the high conservation of the selectivity filter, but also shows that some amino acids in this region are less conserved. The KCNQ1 channel sequence is aligned with related sequences SEQ ID NO: 1-37. The numbers at the top indicate the number of the amino acid residue in the protein sequence. Shaded amino acid residues indicate residues identical to the KCNQ1 channel shown at the bottom of the alignment. The numbers beneath the alignment indicate the adjusted Shannon entropy score of the alignment. A lightly shaded rectangle around this number indicates an adjusted Shannon entropy score in the lower tertile, with medium shading in the middle tertile, and dark shading corresponding to the upper tertile. The mutations present in the study population are depicted as ellipses at the bottom of the figure. The relatively high adjusted Shannon entropy score of 0.72 at residue number 310 (arrow), despite Valine being unique to the KCNQ1 channel, is due to the fact, that Methionine and Leucine account for 36 of 38 residues in the column.

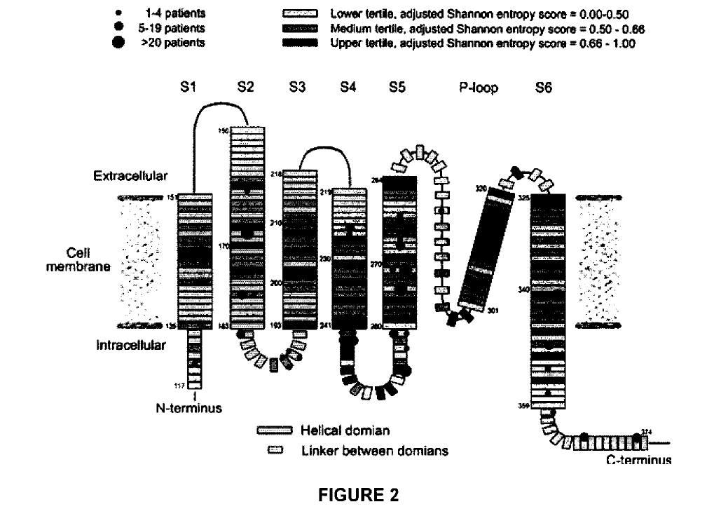

FIG. 2 shows the diversity in conservation between amino acid residues in the investigated regions of the KCNQ1 channel and the location and number of subjects with mutations included in the study. The wide rectangles indicate residues in alpha-helical domains and the small rectangles indicate residues in extracellular and intracellular linkers and in the proximal N- and C-terminus. The shading of the rectangles represent the degree of conservation by the tertile of the adjusted Shannon entropy score, as is shown in the figure. The numbers of subjects carrying each mutation are depicted in the figure by the diameter of the circles. A majority of the mutations are clustered in the S5-pore-S6-region. The phenotype and genotype characteristics of the study population by tertile of the adjusted Shannon entropy score are presented in Table 1.

TABLE I
Baseline Characteristics of Study Population by Tertiles of the Adjusted Shannon Entropy Score

| | Lower Tertile | | Middle Tertile | | Upper Tertile | |
|---|---|---|---|---|---|---|
| N (%) | 146 | (30) | 150 | (30) | 196 | (40) |
| Adjusted Shannon entropy | 0.35 | (0.33-0.40) | 0.51 | (0.51-0.54) | 0.83 | (0.67-0.88) |
| Age at first cardiac event | 13.5 | (8.5-20.0) | 12.0 | (7.0-17.0) | 9.0 | (6.0-12.0) |
| Males, n (%) (unknown, n = 2) | 67 | (46) | 60 | (40) | 85 | (43) |
| Probands, n (%) | 29 | (20) | 21 | (14) | 36 | (18) |
| QTc >500, n (%) | 43 | (30) | 31 | (21) | 66 | (34) |
| QTc, ms | 490 | (460-510) | 460 | (440-490) | 490 | (460-530) |
| Missing ECGs | 34 | (24) | 11 | (8) | 38 | (19) |
| Experienced cardiac event, n (%) | 48 | (33) | 50 | (33) | 125 | (64) |
| Experienced ACA, n (%) | 4 | (3) | 2 | (1) | 5 | (3) |
| Experienced SCD, n (%) | 7 | (5) | 12 | (8) | 29 | (15) |
| First cardiac event type (syncope/ACA/SCD) | 40/4/6 | (27/3/4) | 41/2/7 | (27/2/7) | 110/5/10 | (56/3/5) |
| On beta-blocker therapy, n (%) | 69 | (47) | 56 | (37) | 99 | (51) |
| Age at beta-blocker therapy initiation, years | 12.0 | (7.0-23.0) | 12.9 | (7.0-22.4) | 9.0 | (3.6-22.2) |

| | | | Adjusted | | | Adjusted | | | Adjusted |
|---|---|---|---|---|---|---|---|---|---|
| | | | Shannon | | | Shannon | | | Shannon |
| | Mutation | a | entropy | Mutation | a | entropy | Mutation | a | entropy |
| Mutations included | Y184S | 14 | 0.21 | W120C | 2 | 0.62 | E160K | 3 | 0.80 |
| in the study | R190Q | 4 | 0.40 | G168R | 66 | 0.51 | R174H | 2 | 0.83 |
| shown by tertiles | R190W | 1 | 0.40 | A178P | 5 | 0.54 | R243C | 13 | 0.93 |
| | S225L | 12 | 0.35 | A178T | 1 | 0.54 | R243S | 1 | 0.93 |
| | A226V | 3 | 0.39 | G189E | 2 | 0.52 | V254M | 62 | 0.67 |
| | R259C | 2 | 0.38 | G189R | 4 | 0.52 | E284K | 2 | 0.74 |
| | R259L | 5 | 0.38 | D242N | 3 | 0.52 | W304R | 1 | 0.83 |
| | G269D | 1 | 0.39 | D242Y | 1 | 0.52 | W305C | 3 | 0.75 |
| | G269S | 41 | 0.39 | L266P | 22 | 0.49 | W305S | 12 | 0.75 |
| | L273F | 9 | 0.40 | S277L | 7 | 0.65 | V310I | 2 | 0.72 |
| | I274V | 1 | 0.42 | F296S | 1 | 0.66 | T312I | 17 | 0.84 |
| | Y278H | 2 | 0.35 | A302V | 1 | 0.61 | G314S | 19 | 1.00 |
| | G292D | 3 | 0.23 | G306R | 2 | 0.65 | Y315C | 10 | 0.83 |
| | L353P | 4 | 0.33 | A344V | 18 | 0.57 | Y315S | 1 | 0.83 |
| | Q357H | 3 | 0.41 | S349W | 15 | 0.51 | D317G | 5 | 0.83 |
| | R360G | 3 | 0.35 | | | | T322M | 2 | 0.91 |
| | H363N | 17 | 0.45 | | | | G325R | 8 | 0.83 |
| | R366W | 15 | 0.26 | | | | A141E | 10 | 0.88 |
| | S373P | 6 | 0.18 | | | | A341V | 22 | 0.88 |
| | | | | | | | P343S | 1 | 0.79 |

Numbers in parenthesis indicate percentage or interquartile range. ACA = aborted cardiac arrest; SCD = sudden cardiac death.

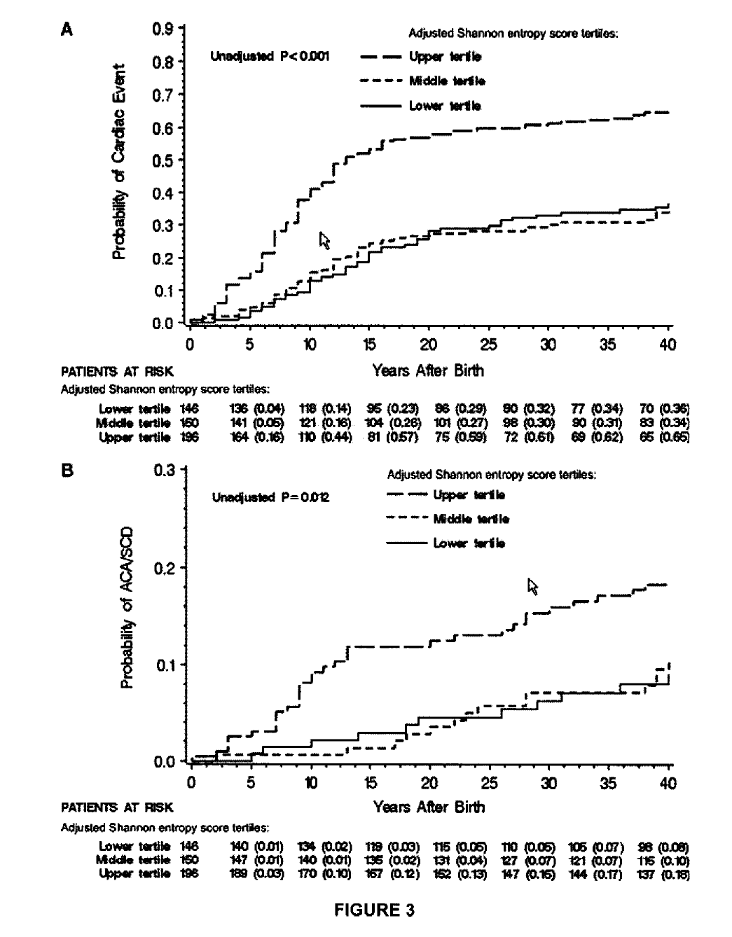

The cumulative age-related probability of a first cardiac event by tertile of the adjusted Shannon entropy score is presented in FIG. 3. The greatest rate of cardiac events is concentrated in the highest tertile. The results of the Cox time-dependent analyses for time to first cardiac event and time to first aborted cardiac arrest/sudden cardiac death are shown in Table 2A and 2B, respectively. The highest tertile is associated with a hazard ratio of 3.32 [2.15-5.13], p<0.001 for first cardiac event and 2.62 [1.06-6.47], p=0.04 for aborted cardiac arrest/cardiac death compared with the lowest tertile, after adjustment for relevant covariates including QTc, age, sex, and beta-blocker therapy. Thus, the risk associated with a mutation in a highly conserved area of the channel is independent of QTc duration. Beta-blocker therapy was associated with a significant decrease in the risk of cardiac events (hazard ratio=0.20, p<0.001), and there were no significant interactions between beta-blocker therapy and other covariates. In the model for aborted cardiac arrest or sudden cardiac death, the adjusted Shannon entropy score is significantly predictive of the endpoint, whereas neither sex nor QTc contributes significantly to the model. Beta-blockers showed a trend towards significance effect in the prevention of aborted cardiac arrest or sudden cardiac death (HR=0.42, p=0.08). Both models were adjusted for patients who died before having an ECG recorded.

TABLE 2
Hazard Ratios for (A) Syncope. Aborted Cardiac
Arrest of Sudden Cardiac Death and (B) Aborted
Cardiac Arrest or Sudden Cardiac Death

| | | 95% Confidence | |
|---|---|---|---|
| Parameter | Hazard Ratio | Limits | P-value |
| (A) | | | |
| QTc >500 ms | 2.30 | 1.66-3.18 | <0.001 |
| Male gender <age 13 | 1.81 | 1.27-2.56 | <0.001 |
| Beta-blocker therapy | 0.20 | 0.11-0.38 | <0.001 |
| Adjusted Shannon | 1.0 | — | — |
| entropy first tertile | | | |
| as reference | | | |
| Second tertile | 1.19 | 0.85-1.67 | 0.42 |
| Third tertile | 3.32 | 2.15-5.13 | <0.001 |
| (B) | | | |
| QTc >500 ms | 2.09 | 0.89-4.93 | 0.09 |
| Male gender <age 14 | 2.04 | 1.02-4.07 | 0.04 |
| Beta-blocker therapy | 0.42 | 0.16-1.11 | 0.08 |
| Adjusted Shannon | 1.0 | — | — |
| entropy first tertile | | | |
| as reference | | | |
| Second tertile | 1.58 | 0.72-3.50 | 0.26 |
| Third tertile | 2.62 | 1.06-6.47 | 0.04 |

### Discussion

Patients with mutations located in highly conserved amino acid residues within the KCNQ1 channel have a higher risk of a first cardiac event and a higher risk of aborted cardiac arrest/sudden cardiac death than patients with mutations in less conserved amino acid residues.

The risk associated with mutation conservation is independent of conventional risk factors such as QTc, sex, and beta-blocker therapy.

Beta-blocker therapy is equally effective in patients with high-risk mutations involving highly conserved amino acid residues as well as in lower risk mutations in less conserved amino acid residues.

In this study, the only parameter significantly associated with aborted cardiac arrest and/or cardiac death was the adjusted Shannon entropy score of the mutation location.

Mutations in the KCNQ1 channel can lead to very different clinical syndromes dependent on the location of the mutation. Missense mutations can cause a loss of the channel function resulting in LQT1 syndrome, or can cause a gain-of function causing either atrial fibrillation¹⁹ or short QT syndrome.²⁰ Interestingly mutations causing atrial fibrillation have been described mostly in the S1 subunit,^{19, 21, 22} and LQTS-causing mutations have been described throughout the KCNQ1 protein, but seem to cluster in the S5-pore-S6 region and the intracellular linkers. Moss et al⁸ and Shimuzu et al²³ have shown a higher event rate in patients with mutations located in the transmembrane region of the channel compared to mutations located in the N-terminus or C-terminus domains, and one individual mutation has been associated with a severe clinical course.^{24, 25} In this study, it is demonstrated for the first time, that high-risk mutations are located in conserved amino acid residues within the channels, and that these mutations can be identified easily using a readily available bio-informational analysis method.

How mutations in conserved amino acid residues can cause a more virulent clinical course is still unknown, but the impact on channel function by different mutations has been described previously. Mutations causing haploinsufficiency are associated with a less severe clinical course than mutations causing dominant-negative electrophysiological effects.^{8, 26} Information on channel function for mutations included in this study was too sparse to allow us to investigate this matter in the present study, since only five of the included mutations have been characterized as causing haploinsufficiency and were distributed in all three adjusted Shannon entropy score groups. Other proposed methods of channel dysfunction are interruption of regions involved in protein interaction²⁷ and alteration of channel gating kinetics.²⁸ Interestingly, in the last study, a row of neighboring amino acid residues, residue number 348 through 362, were tested for influence on function. Only two amino acid residues, F351 and V355, were reported to be important for channel activation, and both have very high conservation scores (adjusted Shannon entropy=0.81 and 0.87, respectively) compared to the neighboring amino acid residues (FIG. 1). From these data, one may speculate that conservation scoring identifies amino acid residues of high functional importance, and that interruption of these important channel functions adds further substrate for development of arrhythmias beyond a simple decrease in the I_{Ks} current.

Only studied missense mutations in the main body of the channel have been studied and excluded mutations in the distal parts of the N- and C-terminus have been excluded. However, the risk of cardiac events in subjects with non-missense mutations and mutations outside in the distal regions of the N- and C-terminus, were not significantly different from the risk of subjects in the lower- and middle adjusted Shannon entropy score groups. Missense mutations are the most frequent type mutation causing the LQT1, and most missense mutations are located in the transmembrane region.²⁹ Therefore, the findings within this study should be applicable to most LQT1 patients.

Several more sophisticated methods for scoring amino acid conservation have been developed^{16, 30-33} involving the propensity of amino acid residues, the entropy of neighboring amino acid residues and 3-dimensional molecular structure. Most of these approaches are extensions of the Shannon entropy and a variety of these methods have been reviewed by Valdar.¹⁴ No method has ever been applied for risk stratification in clinical medicine, and presumptions of how important amino acid residues are in regard to the structure and function of a mutated ion channel, and especially in regard to the associated clinical risk, would be purely speculative. Ahola et al³⁴ compared five different conservation scores, among these the Shannon entropy, and found that all were equally comparable in finding conserved amino acid residues within proteins. Fischer et al³¹ tested 4 methods and confirmed these findings. Four methods were applied: the Shannon entropy, the Von Neumann entropy, the Property entropy, and the Jensen-Shannon diversity, all available in the conservation scoring tool by Capra et al.¹⁶ The results were very similar and resulted in an almost identical classification of the mutations as highlighted in the current study.

### Limitations

The Shannon entropy score is linked to family membership of the study subjects and is likely to be influenced by other genotypic traits in the family. Several methods were used to test for statistical robustness in order to investigate the influence of family size and family membership on the data, and little or no confounding was found. When weighing the influence of each family with the inverse of the family size, the hazard ratios for the adjusted Shannon entropy score increased. Any errors due to family size and family memberships are likely to be small.

The outcome analyses included subjects from families with a known KCNQ1 mutation who died suddenly and unexpectedly at a young age and were classified as LQTS-related death with the same mutation that was present in the family. It is possible that a few of these subjects could have died from a non-LQTS cause or had an LQTS mutation different from the family mutation, but that is unlikely

### CONCLUSION

The degree of conservation of individual amino acids in the KCNQ1 channel can be scored using bio-informational analysis, and the degree of conservation predicts the severity of the clinical course in patients with missense mutations in the KCNQ1 channel. The associated risk is independent of other significant risk factors such as QTc duration, sex, and beta-blocker therapy.

### Example 2

### Risk Stratification in Cystic Fibrosis

### Background

Cystic fibrosis (CF) is a monogenetic disorder caused by mutations in the CFTR gene. This gene encodes for a chloride ion channel located in several organs in the human body, but being especially important in the lungs and pancreas, where it is crucial for lung resistance to infections and normal pancreatic secretion of digestive enzymes. The disease is recessive meaning that affected patients have mutations in both the genes present in the human DNA. Subjects with mutations in only one of the genes have no symptoms, but can pass the mutation on to offspring. More than 1000 mutations are known today, but one mutation is found in 70% of affected patients and 10-20 other mutations account for additional 10-15% of patients³⁵.

Mutations in the CFTR genes can cause a number of different types of alteration in gene expression, function and availability depending on location and the type of mutations. Even within each type of mutation, the clinical course varies considerably³⁵. There is no uniformly accepted classification of the severity of CF, but the pancreas is the most frequently affected organ in CF (affected in 85% of patients) and it is feasible to divide the CF population into patients requiring pancreatic enzyme supplementation, i.e. pancreatic insufficiency (PI), and patients with a sufficient pancreatic function to sustain normal digestive function without digestive enzyme supplementation (PS)³⁶. Using this classification, the mutations can be divided into severe or mild mutations depending on the degree of pancreatic affection the mutation have been observed to cause. A presence of one or two mild mutations are associated with a mild phenotype, while it takes two severe mutations, i.e. one in each allele, to cause the severe phenotype. This classification of patients has been proven to show a good overall correlation with the general clinical course of the patients, and is used to decide whether aggressive early treatment should be given to newborns with two severe mutations³⁶.

### Methods

The CFTR (SEQ ID NO:39) channel is a member of the ABCC ion channel family. The sequences of the thirteen members of the ABCC ion channel family were drawn from the Uniprot database³⁷ and aligned using the public sequence aligner CLUSTALW2³⁸.

Information on phenotype-genotype correlation, i.e. the severity of the clinical course of patients who have been diagnoses with the mutation, was drawn from the “Cystic Fibrosis Mutation Database” found at http://www3.genet.sickkids.on.ca and from the paper by Kristidis et al³⁹. Because CF is a recessive disease, mutations in both alleles are necessary to cause the disease. The mutations where divided into “severe” if the mutation is known to cause PI and severe lung disease, “mild”, for mutations that were associated with both PS and mild to moderate lung disease and a known ‘severe’ mutation was present in the other allele, and “intermediate” if it is associated with either PS or mild lung disease and a ‘severe’ mutation was present in the other allele. Intermediate mutations were not included in this study. This study concerned only missense mutations and single point deletions.

The adjusted Shannon entropy was calculated for all included mutations (see Example 1). The baseline values for each phenotype classification are presented as median [Interquartile range]. The groups were compared using wilcoxon rank sum test and odds-ratio. A p-value of less than 0.05 was considered significant.

### Results

Information on phenotype-genotype correlation was found for 59 mutations. 26 were classified as mild, 7 as intermediate and 26 as severe. The included mutations, the classification and the adjusted Shannon entropy for each mutation are shown in Table 3. The median adjusted Shannon entropy were significantly different between the “mild” mutations group (median=0.54 [0.45-0.63]) and the “severe” group (median=0.83 [0.76-0.89]), p<0.0001. Using a similar cutoff-point as in Example 1, a mutation with an adjusted Shannon entropy higher than 0.67 has a high risk of being a severe mutation (OR=11.4 [3.1-42.0], p<0.0001).

TABLE 3
Included CFTR mutations and corresponding
values for the adjusted Shannon entropy.

| Mild | ASE | Severe | ASE |
|---|---|---|---|
| V754M | 0.08 | D985Y | 0.45 |
| K166Q | 0.30 | H1085R | 0.53 |
| N66S | 0.32 | S1255P | 0.56 |
| s1235R (ref1) | 0.35 | A46D | 0.58 |
| D993G | 0.35 | Q179K | 0.65 |
| R334W | 0.37 | I507del | 0.69 |
| R75L | 0.37 | L102P | 0.69 |
| A309D | 0.38 | D614Y | 0.74 |
| D110E | 0.42 | V562I | 0.74 |
| I980K | 0.43 | A72D | 0.79 |
| R117H | 0.45 | R1066C | 0.82 |
| S108F | 0.46 | L453del | 0.82 |
| H620Q | 0.47 | V520F | 0.86 |
| E193K | 0.48 | F508del | 0.91 |
| R347H/R347P | 0.53 | T501A | 0.91 |
| A566T | 0.58 | L571S | 0.91 |
| D513G | 0.60 | A1067D | 0.91 |
| V1008D | 0.61 | N1303H | 0.92 |
| P205S | 0.66 | S549N | 1 |
| A455E | 0.67 | G480C | 1 |
| D1152H | 0.70 | s549r/s549n | 1 |
| G27E | 0.74 | D579A | 1 |
| L1335P | 0.76 | A559E | 1 |
| P99L | 1 | G551D | 1 |
| G551S | 1 | R560T | 1 |
| P574H | 1 | G480C | 1 |

### CONCLUSION

Mutations in conserved amino acid residues within the CFTR gene have a very high probability of causing pancreatic insufficiency and to cause a severe phenotype if present in both alleles (odds ratio=11.4). Measuring the conservation of amino acid residues in the CFTR gene can be used to risk stratify patients with newly discovered mutations.

### REFERENCES

1. Sanguinetti M C. Long QT syndrome: ionic basis and arrhythmia mechanism in long QT syndrome type 1. J Cardiovasc Electrophysiol 2000; 11 (6):710-712.
2. Wang Q, Curran M E, Splawski I et al. Positional cloning of a novel potassium channel gene: KVLQT1 mutations cause cardiac arrhythmias. Nat Genet 1996; 12(1):17-23.
3. Peroz D, Rodriguez N, Choveau F, Baro I, Merot J, Loussouarn G. Kv7.1 (KCNQ1) properties and charmelopathies. J Physiol 2008; 586(7):1785-1789.
4. Long S B, Campbell E B, MacKinnon R. Crystal structure of a mammalian voltage-dependent Shaker family K+ channel. Science 2005; 309(5736):897-903.
5. Smith J A, Vanoye C G, George A L, Jr., Meiler J, Sanders CR. Structural models for the KCNQ1 voltage-gated potassium channel. Biochemistry 2007; 46(49):14141-14152.
6. Jespersen T, Grunnet M, Olesen S P. The KCNQ1 potassium channel: from gene to physiological function. Physiology (Bethesda)) 2005; 20:408-416.
7. Moss A J. Long QT Syndrome. JAMA 2003; 289(16):2041-2044.
8. Moss A J, Shimizu W, Wilde A A et al. Clinical aspects of type-1 long-QT syndrome by location, coding type, and biophysical function of mutations involving the KCNQ1 gene. Circulation 2007; 115(19):2481-2489.
9. Priori S G, Napolitano C, Schwartz P J et al. Association of long QT syndrome loci and cardiac events among patients treated with beta-blockers. JAMA 2004; 292(11):1341-1344.
10. Zareba W, Moss A J, Daubed J P, Hall W J, Robinson J L, Andrews M. Implantable cardioverter defibrillator in high-risk long QT syndrome patients. J Cardiovasc Electrophysiol 2003; 14(4):337-341.
11. Priori S G, Schwartz P J, Napolitano C et al. Risk stratification in the long-QT syndrome. N Engl J Med 2003; 348(19):1866-1874.
12. Hobbs J B, Peterson D R, Moss A J et al. Risk of aborted cardiac arrest or sudden cardiac death during adolescence in the long-QT syndrome. JAMA 2006; 296(10):1249-1254.
13. Shenkin P S, Erman B, Mastrandrea L D. Information-theoretical entropy as a measure of sequence variability, Proteins 1991; 11(4):297-313.
14. Valdar W S. Scoring residue conservation. Proteins 2002; 48(2):227-241.
15. C. E. Shannon. A Mathematical Theory of Communication. Bell System Technical Journal 1948; 27:379-423.
16. Capra J A, Singh M. Predicting functionally important residues from sequence conservation. Bioinformatics 2007; 23(15):1875-1882.
17. Chenna R, Sugawara H, Koike T et al. Multiple sequence alignment with the Clustal series of programs. Nucleic Acids Res 2003; 31(13):3497-3500.
18. Lin D Y, Wei U. The Robust Inference for the Proportional Hazards Model. Journal of the American Statistical Association 1989; 84(408):1074-1078.
19. Chen Y H, Xu S J, Bendahhou S et al. KCNQ1 gain-of-function mutation in familial atrial fibrillation. Science 2003; 299(5604):25′-254.
20. Bellocq C, van Ginneken A C, Bezzina C R et al. Mutation in the KCNQ1 gene leading to the short QT-interval syndrome. Circulation 2004; 109(20):2394-2397.
21. Ellinor P T, Moore R K, Patton K K, Ruskin J N, Pollak M R, Macrae C A. Mutations in the long QT gene, KCNQ1, are an uncommon cause of atrial fibrillation. Heart 2004; 90(12):1487-1488.
22. Hong K, Piper DR, az-Valdecantos A et al. De novo KCNQ1 mutation responsible for atrial fibrillation and short QT syndrome in utero. Cardiovasc Res 2005; 68(3):433-440.
23. Shimizu W, Horie M, Ohno S et al. Mutation site-specific differences in arrhythmic risk and sensitivity to sympathetic stimulation in the LQT1 form of congenital long QT syndrome: multicenter study in Japan. J Am Coll Cardiol 2004; 44(1):117-125.
24. Brink P A, Crotti L, Corfield V et al. Phenotypic variability and unusual clinical severity of congenital long-QT syndrome in a founder population. Circulation 2005; 112(17):2602-2610.
25. Crotti L, Spazzolini C, Schwartz P J et al. The common long-QT syndrome mutation KCNQ1/A341V causes unusually severe clinical manifestations in patients with different ethnic backgrounds: toward a mutation-specific risk stratification. Circulation 2007; 116(21):2366-2375.
26. Roden D M. Defective ion channel function in the long QT syndrome: multiple unexpected mechanisms. J Mol Cell Cardiol 2001; 33(2):185-187.
27. Howard R J, Clark K A, Holton J M, Minor D L, Jr. Structural insight into KCNQ (Kv7) channel assembly and channelopathy. Neuron 2007; 53(5):663-675.
28. Boulet I R, Labro A J, Raes A L, Snyders D J. Role of the S6 C-terminus in KCNQ1 channel gating. J Physiol 2007; 585(Pt 2):325-337.
29. Tester D J, Will M L, Haglund C M, Ackerman M J. Compendium of cardiac channel mutations in 541 consecutive unrelated patients referred for long QT syndrome genetic testing. Heart Rhythm 2005; 2(5):507-517.
30. Caffrey D R, Somaroo S, Hughes J D, Mintseris J, Huang E S. Are protein-protein interfaces more conserved in sequence than the rest of the protein surface? Protein Sci 2004; 13(1):190-202.
31. Fischer J D, Mayer C E, Soding J. Prediction of protein functional residues from sequence by probability density estimation. Bioinformatics 2008; 24(5):613-620.
32. Landgraf R, Xenarios I, Eisenberg D. Three-dimensional cluster analysis identifies interfaces and functional residue clusters in proteins. J Mol Biol 2001; 307(5):1487-1502.
33. Li X, Kahveci T. A Novel algorithm for identifying low-complexity regions in a protein sequence. Bioinformatics 2006; 22(24):2980-2987.
34. Ahola V, Aittokallio T, Uusipaikka E, Vihinen M. Statistical methods for identifying conserved residues in multiple sequence alignment. Stat Appl Genet Mol Biol 2004; 3:Article 28.
35. Proesmans M, Vermeulen F, De B K. What's new in cystic fibrosis? From treating symptoms to correction of the basic defect. Eur J Pediatr 2008.
36. Zielenski J. Genotype and phenotype in cystic fibrosis. Respiration 2000; 67(2):117-133.
37. The universal protein resource (UniProt). Nucleic Acids Res 2008; 36 (Database issue):D190-D195.
38. Chenna R, Sugawara H, Koike T et al. Multiple sequence alignment with the Clustal series of programs. Nucleic Acids Res 2003; 31(13):3497-3500.
39. Kristidis et al. (1992) Am J Hum Genet. 50(6) 1178-84.
40. Spruance, S L, Reid, J E, Grace, M and Samore, M, Hazard Ratio in Clinical Trials. Antimicrobial Agents and Chemotherapy, 2004; 48(8):2787-2792.

What is claimed is:

1. A database for correlating the risk of developing a disorder in a subject with the presence of a mutation in a protein sequence, the database being recorded on a computer readable medium and constructed by a method comprising:
obtaining a plurality of protein sequences of related proteins from the subject;
aligning the protein sequences;
determining the conservation scores for the amino acids of the protein sequences; and
identifying individual protein sequences having mutations at an amino acid residue with a high conservation score;
wherein proteins sequences having mutations at an amino acid residue with a high conservation score are associated with an increased risk for the subject of developing the disorder.

2. The database of claim 1, wherein the conservation score is determined using the adjusted Shannon entropy of the amino acid residue.

3. The database of claim 1, wherein the number of related proteins is about 12 or more.

4. The database of claim 2, wherein a high conservation score is an adjusted Shannon entropy score of about 0.5 or more.

5. The database of claim 2, wherein a high conservation score is an adjusted Shannon entropy score of about 0.66 or more.

6. A method for determining the risk of a subject of developing a disorder comprising:
obtaining a body fluid or tissue sample from a patient;
isolating nucleic acid from the body fluid or tissue sample;
obtaining a sample protein sequence information for a protein of interest from the subject by sequencing the region of the nucleic acid encoding the protein of interest;
determining the conservation score for each amino acid in the sample protein sequence by comparison to a database on a computer readable medium containing a plurality of related protein sequences; and
determining if the sample protein sequence has mutated amino acids at positions with high conservation scores;
wherein proteins sequences having mutations at an amino acid residue with a high conservation score are associated with an increased for the subject of developing the disorder.

7. The method of claim 6, wherein the body fluid or tissue sample is selected from the group consisting of: blood, saliva and cells.

8. The method of claim 6, wherein the conservation score is determined using the adjusted Shannon entropy of the amino acid residue.

9. The method of claim 6, wherein the number of related proteins is about 12 or more.

10. The method of claim 8, wherein a high conservation score is an adjusted Shannon entropy score of about 0.5 or more.

11. The method of claim 8, wherein a high conservation score is an adjusted Shannon entropy score of about 0.66 or more.

12. A method for determining the risk of a subject of developing a disorder comprising:
obtaining a sample protein sequence information for a protein of interest from the subject;
determining the conservation score for each amino acid in the sample protein sequence by comparison to a database on a computer readable medium containing a plurality of related protein sequences;
classifying the conservation scores into strata defined by ranges of conservation score values;
determining if the sample protein sequence has mutated amino acids having a conservation score in one of the strata; and
correlating the strata with an increased risk of developing the disorder; wherein the strata having the highest conservation score range is associated with the highest risk of developing the disorder.

13. The method of claim 12, wherein there are between 3 and 10 strata.

14. The method of claim 13, wherein there are three strata.

15. The method of claim 14, wherein the conservation score ranges for the strata are:
1^{st} stratum: 0.00-about 0.50;
2^{nd} stratum: about 0.50-about 0.66; and
3^{rd} stratum: about 0.66-1.00.

16. A method for determining the hazard ratio for a subject of developing a disorder comprising:
obtaining a sample protein sequence information for a protein of interest from the subject;
determining the conservation score for each amino acid in the sample protein sequence by comparison to a database on a computer readable medium containing a plurality of related protein sequences;
classifying the conservation scores into strata defined by ranges of conservation score values; wherein each stratum is associated with a hazard ratio for developing the disorder;
determining if the patient has a mutation at an amino acid in the protein of interest;
obtaining the conservation score for the amino acid that is mutated in the subject; and
correlating the conservation score for the mutated amino acid with the hazard ratio for that conservation score; wherein the hazard ratio for the conservation score is the hazard ratio for the subject for developing the disorder.
